(19) United States
(12) Patent Application Publication
(10) Pub. No.: US 2016/0303137 A1
(43) Pub. Date: Oct. 20, 2016

(54) DUAL PI3K AND WNT PATHWAY INHIBITION AS A TREATMENT FOR CANCER

(71) Applicant: Indiana University Research and Technology Corporation, Indianapolis, IN (US)

(72) Inventors: Milan Radovich, Zionsville, IN (US); Jeffrey Peter Solzak, Indianapolis, IN (US)

(21) Appl. No.: 15/133,443

(22) Filed: Apr. 20, 2016

Related U.S. Application Data
(60) Provisional application No. 62/149,787, filed on Apr. 20, 2015.

(51) Int. Cl. A61K 31/5377; A61K 31/497

(52) U.S. Cl. CPC A61K 31/5377; A61K 31/497

(57) ABSTRACT

Disclosed is a combination therapy for cancer. Additionally, the administration of inhibitors of the phosphoinositide 3-kinase (PI3K) signaling pathway and the Wnt signaling pathway are disclosed for treatment of cancer, and in particular, triple-negative breast cancer (TNBC).

### CROSS-REFERENCE TO RELATED APPLICATIONS

This application claims priority to U.S. Provisional Patent Application No. 62/149,787 filed on Apr. 20, 2015, which is hereby incorporated by reference in its entirety.

### BACKGROUND OF THE DISCLOSURE

The present disclosure relates generally to a combination therapy for cancer. More particularly, the present disclosure is directed to administering inhibitors of the phosphoinositide 3-kinase (PI3K) signaling pathway and the WNT signaling pathway for treatment of cancer.

Cancers are a large family of diseases that involve abnormal cell growth with the potential to invade or spread to other parts of the body. Cancer tumors contain groups of cancer cells often in the form of a mass or lump, but may be distributed diffusely. More than one million people in the United States get cancer each year.

Triple negative breast cancer (TNBC), in particular, accounts for 15% of all breast cancer cases in the United States, and despite its lower incidence, contributes to a disproportionately higher rate of morbidity and mortality compared to other breast cancer subtypes. Studies have shown that TNBC is more likely to spread beyond the breast and more likely to recur after treatment. For example, a study of more than 1,600 women in Canada published in 2007 found that women with TNBC were at higher risk of having the cancer recur outside the breast—but only for the first 3 years. Other studies have reached similar conclusions. As years go by, the risks of TNBC recurring become similar to those risk levels for other types of breast cancer.

It is believed that most triple-negative breast cancers are of the basal-like cell type. “Basal-like” means that the cells resemble the basal cells that line the breast ducts. Like other types of breast cancer, basal-like cancers can be linked to family history, or they can happen without any apparent family link. Basal-like cancers tend to be more aggressive, higher grade cancers (higher the grade, the less the cancer cells resemble normal, healthy cells in their appearance and growth patterns).

Because these tumors lack expression of three receptors known to fuel most breast cancers, estrogen (ER-), progesterone (PR-), or human epidermal growth factor receptor 2 (HER-2) receptors (“triple negative”); that is, the growth of the tumors is not supported by the hormones estrogen and progesterone, nor by the presence of too many HER2 receptors, triple-negative breast cancer does not respond to hormonal therapy (such as tamoxifen or aromatase inhibitors) or therapies that target HER2 receptors, such as Herceptin (chemical name: trastuzumab).

Accordingly, there is a need in the art to improve the outcomes of cancer patients through the implementation of targeted agents. Particularly, it would be beneficial if there was a drug therapy that would provide an anti-tumor effect against cancers such as TNBC, glioblastoma, prostate cancer, non-small cell lung cancer, colon cancer, melanoma, esophageal cancer, and ovarian cancer.

### BRIEF DESCRIPTION OF THE DISCLOSURE

The present disclosure is generally related to a combination of small molecule inhibitors that inhibit two major canonical pathways for treating cancer. More particularly, the present disclosure is related to combination therapy including an inhibitor of the phosphoinositide 3-kinase (PI3K) signaling pathway and an inhibitor of the Wnt signaling pathway for treating cancer. In one particularly suitable embodiment, the cancer is triple-negative breast cancer (TNBC).

Accordingly, in one aspect, the present disclosure is directed to a composition including a phosphoinositide 3-kinase (PI3K) inhibitor and a Wnt inhibitor.

In another aspect, the present disclosure is directed to a method for treating cancer in a subject in need thereof. The method includes administering a composition comprising a phosphoinositide 3-kinase (PI3K) inhibitor and a Wnt inhibitor to the subject.

In yet another aspect, the present disclosure is directed to a method for treating triple-negative breast cancer in a subject in need thereof. The method includes administering a composition comprising a phosphoinositide 3-kinase (PI3K) inhibitor and a Wnt inhibitor to the subject.

In yet another aspect, the present disclosure is directed to a method for reducing cancer cell growth in a subject in need thereof. The method includes administering a composition comprising a phosphoinositide 3-kinase (PI3K) inhibitor and a Wnt inhibitor to the subject.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts an Ingenuity Pathway Analysis (IPA) Molecular Activity Pathway analysis, predicting the effects of adding the PI3K inhibitor Buparlisib (BKM120) in silico (red) on the Wnt pathway. When Buparlisib (BKM120) is added, inhibition of PI3K pathway components is observed as expected, but there is also a resulting activation of the Wnt pathway, including Wnt ligands, Frizzled receptors, and Beta-Catenin (CTNNB 1).

FIG. 2A depicts a Pathway analysis of RNA-seq data comparing 94 TNBCs versus 20 normal breast tissues, The analysis revealed the overexpression of the PI3K/AKT pathway. Statistical analysis revealed 3,197 genes (FDR <5%, with a fold change more than ±2) that are differentially expressed between TNBC and normal. Significant genes were imported into Ingenuity Pathway Analysis (FIG. 1), revealing an active PI3K/AKT pathway.

FIG. 2B depicts a TCGA analysis using the cBioPortal database of 139 TNBC patients. The analysis showed that approximately 92% of individuals have an observed genomic aberration within the PI3K/AKT pathway. In this oncoprint plot, each row represents the stated gene, and each column is an individual patient. A key representing each type of aberration is depicted below the plot.

FIG. 3A depicts the RNA expression of Wnt pathway molecules after Buparlisib treatment of the TNBC cell line MDA-MB-231. Cells were treated with Buparlisib and RNA-seq (Ampliseq Transcriptome, see methods) was performed on extracted RNA before and after treatment. Genes in the WNT pathway that were observed to have a change in expression include the protein target of WNT974, Porcupine (PORCN), as well as the Wnt ligands, Frizzled (FZD) receptors, PTK7, LRP4/6, and Beta-Catenin (CTNNB1).

FIG. 3B depicts a Western blot of Porcupine (PORCN), phosphorylated-AKT (p-AKT Ser 473), total AKT, and Beta-Actin (loading control), before and after treatment of 3 TNBC cell lines with Buparlisib. The western blot shows induction of Porcupine protein after treatment with Buparlisib in all three cell lines.

FIGS. 4A-4C depict the in vitro efficacy of Buparlisib and WNT974 across three TNBC cell lines. Buparlisib and WNT974 were dosed at increasing concentrations (x & y-axes), and the percentage of cell viability was measured (z-axis) for cell lines: MDA-MB-231 (FIG. 4A), Hs578T (FIG. 4B) and HCC70 (FIG. 4C). Data is displayed as a cascade plot. Using the Chou-Talalay method, a ˜50% reduction was observed in cell viability at 100 nM concentration of each drug for MDA-MB-231 and Hs578T (Combination Index=0.33, and 0.36 respectively). For HCC70, an additive effect was observed that was more resistant to the combination with an IC50 of 1 μM.

FIGS. 5A & 5B depict a pharmacodynamic (PD) study of Buparlisib+WNT974 using cell line xenografts of the TNBC cell line, TMD231 (a MDA-MB-231 cell line variant that metastasizes to the lung). FIG. 5A is a graph of tumor volumes of NSG mice implanted with TMD-231 cells and treated with Vehicle, Buparlisib, WNT974, or the combination. After 7 days of treatment, a ˜40% decrease was observed in tumor volume. FIG. 5B depicts the quantification of drug in tumors resected from mice treated in the PD study. Half of the tumors were resected at 1 hr-post the last dose, and the other half Thr-post the last dose. Using HPLC-MS/MS, Buparlisib and WNT974 were quantified as nanograms of drug per gram of tumor tissue (ng/g). Average values with standard deviation is presented. LOQ=Limit of Quantitation.

FIGS. 6A & 6B depict the in vivo biomarker results on TMD-231 tumors in the pharmcodynamics study. FIG. 6A depicts pAKT blotting observed expression in the vehicle and WNT974 groups and a lack of expression in Buparlisib and combination groups, demonstrating potent PI3K inhibition. FIG. 6B depicts that Axin2 RNA expression displayed no significant change in the vehicle and Buparlisib groups, but saw a decrease in WNT974 and combination groups, indicating WNT974 activity.

FIG. 7 shows the effect of the combination therapy on the growth of lung metastases. When compared with the control group, Buparlisib and WNT974 as single agents displayed small, but non-significant, decreases in lung metastases. The combination treatment, however, resulted in a significant decrease in lung metastatic burden compared to the control group (P=0.0196).

FIG. 8 depicts NSG mice that were implanted with a TNBC PDX and were treated in four groups (n=10 each): vehicle, Buparlisib, WNT974, and the combination of Buparlisib+WNT974. At the end of the experiment, 80% of mice treated with the combination survived to the end of the study. In comparison, only 40%, 30%, and 0% of mice survived treated with Buparlisib, Vehicle, and WNT974, respectively.

FIG. 9 depicts the TNBC PDX study. Over the course of the 28 day study, there was no significant change in body weight among surviving mice.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Generally, the present disclosure is directed to a combination treatment for cancers, and in particular, triple-negative breast cancer. Particularly, a composition is provided including inhibitors of two major canonical pathways that have now been shown to combine to provide an anti-tumor effect in triple-negative breast cancer cells. The first inhibitor of the composition is an inhibitor of the phosphoinositide 3-kinase (PI3K) signaling pathway, and the second inhibitor of the composition is an inhibitor of the Wnt signaling pathway. It should be understood, however, that more than two inhibitors of these pathways can be used in the composition of the present disclosure.

Phosphatidylinositol 3-kinases (PI3Ks) are lipid kinases that play a central role in the regulation of cell cycle, apoptosis, DNA repair, senescence, angiogenesis, cellular metabolism, and motility. They act as intermediate signaling molecules and are most well known for their roles in the PI3K/AKT/mTOR signaling pathway. PI3Ks transmit signals from the cell surface to the cytoplasm by generating second messengers—phosphorylated phosphatidylinositols—which in turn activate multiple effector kinase pathways, including tyrosine-protein kinase (BTK), protein kinase B (AKT), protein kinase C (PKC), nuclear factor kappa-light-chain-enhancer of activated B cells (NF-kappa-B), and c-jun N-terminal kinase/stress-activated protein kinase (JNK/SAPK) pathways, and ultimately result in survival and growth of normal cells. Although the activity of PI3Ks is tightly regulated in normal cells by internal signals such as PTEN (phosphatase and tensin homolog deleted from chromosome 10), it has been recognized that deregulation of the PI3K signaling pathway is associated with development in one-third of human cancers. Aberrantly activated PI3K pathway promotes carcinogenesis and tumor angiogenesis. For example, approximately 30% of breast cancers demonstrated activating missense mutations of PIK3CA, the gene encoding the catalytic p110a subunit of class I PI3K, and the mutated gene provides cells with a growth advantage and promotes tumorigenesis. In addition, dysregulated PI3K pathway signaling has been implicated in conferring resistance to conventional therapies including biologics, hormonal therapy, tyrosine kinase inhibitors, radiation, and cytotoxics in breast cancer, glioblastoma, and non-small cell lung cancer. Other genetic aberrations that drive the PI3K pathway in cancer include gene amplification of PI3Ks, loss of the regulatory activity of PTEN, and activating mutations of receptor tyrosine kinases (RTKs) such as epidermal growth factor receptor (EGFR) and human epidermal growth factor receptor 2 (HER2). PI3Ks are enzymes of approximately 200-300 kDa in molecular weight. In human, three distinct classes of PI3Ks (I-III) have been identified.

Further, PI3K inhibitors are divided into three classes, pan-class I, isoform-selective and dual PI3K/mTOR inhibitors, based on pharmacokinetic properties and isoform selectivity for the ATP binding site of PI3Ks. It has been found that PI3K inhibitors, and particularly, pan-PI3K inhibitors, can be used in the compositions of the present disclosure. Suitable PI3K inhibitors include, for example, 5-(2,6-di-4-morpholinyl-4-pyrimidinyl)-4-(trifluoromethyl)-2-pyridinamine (buparlisib; B KM-120); 2-(1H-indazol-4-yl)-6-[[4-(methylsulfonyl)-1-piperazinyl]methyl]-4-(4-morpholinyl)-thieno [3 ,2-d]pyrimidine (Pictilisib, GDC-0941); (1E,4S,4aR,5R,6aS,9aR)-5-(acetyloxy)-1-[(di-2-propen-1-ylamino)methylene]-4,4a,5,6,6a, 8,9,9a-octahydro-11-hydroxy-4-(methoxymethyl)-4a,6a-dimethyl-cyclopenta[5 ,6]naphtho[1,2-c]pyran-2,7,10(1H)-trione (PX866); 2-{3-[2-(1-isopropyl-3-methyl-1H-1,2-4-triazol-5-yl)-5,6-dihydrobenzo[f]imidazo[1,2-d][1,4]oxazepin-9-yl]-1H-pyrazol-1-yl}-2-methylpropanamide (taselisib; GDC-0032); 2-methyl-2-[4-(3-methyl-2-oxo-8-quinolin-3-ylimidazo[4,5-c]quinolin-1-yl)phenyl]propanenitrile (dactoblisib; BEZ-235); (2S)-N1-[4-methyl-5-[2-(2,2,2-trifluoro-1,1-dimethylethyl)-4-pyridinyl]-2-thiazolyl]-1,2-pyrrolidinedicarboxamide (alpelisib; BYL-719); (Z)-but-2-enedioic acid; 8-(6-methoxypyridin-3-yl)-3-methyl-1-[4-piperazin-1-yl-3-(trifluoromethyl)phenyl]imidazo[4,5-c]quinolin-2-one (BGT-226); (2S)-1-[4-[[2-(2-aminopyrimidin-5-yl)-7-methyl-4-morpholin-4-ylthieno[3,2-d]pyrimidin-6-yl]methyl]piperazin-1-yl]-2-hydroxypropan-1-one (apitolisib; GDC-0980); N-[4-[[[3-[(3,5-dimethoxyphenyl)amino]-2-quinoxalinyl]amino]sulfonyl]phenyl]-3-methoxy-4-methyl-benzamide (voxtalisib; XL-765); 2-amino-N-[3-[[3-(2-chloro-5-methoxyanilino)quinoxalin-2-yl]sulfamoyl]phenyl]-2-methylpropanamide (pilaralisib; XL-147); 3-(2,4-diaminopteridin-6-yl)phenol (TG100713); 1-[4-(3-ethyl-7-morpholin-4-yltriazolo[4,5-d]pyrimidin-5-yl)phenyl]-3-[4-(4-methylpiperazine-1-carbonyl)phenyl]urea (PKI-402); 5-Fluoro-3-phenyl-2-[(1S)-1-(7H-purin-6-ylamino)propyl]-4(3H)-quinazolinone (idelalisib); and combinations thereof. In one embodiment, the composition includes the PI3K inhibitor, 5-(2,6-di-4-morpholinyl-4-pyrimidinyl)-4-(trifluoromethyl)-2-pyridinamine (buparlisib; BKM-120).

Proper dosing can be determined by those skilled in the art. As understood by those skilled in the art, dosing can depend on the severity of disease, route of administration, subject body type, subject body weight, age of the subject, and combinations thereof. For example, an initially high dose can be administered to a subject that is subsequently reduced to reduce side-effects and adverse effects of the PI3K inhibitor. Alternatively, a low dose can be initially administered to a subject that is subsequently increased to a dosage resulting in the reduction of tumor size, reduction of tumor growth rate, reduction in cell migration, reduction of cell proliferation, and combinations thereof. For orally administered buparlisib, for example, 100 mg/day can be administered. As described above, the dosage of orally administered buparlisib can be increased or decreased from 100 mg/day depending on the response of the tumor growth and symptoms associated with adverse and/or side-effects. In one embodiment, a single dosage of orally administered buparlisib is from about 3 mg/kg to about 30 mg/kg. In other embodiments, GDC0941 is administered orally in an amount of about 60 mg/day; PX866 is orally administered in an amount of about 8 mg/day; GDC0032 is orally administered in an amount of about 4 mg/day; BYL-719 is orally administered in an amount of about 250 mg/day; GDC-0980 is orally administered in an amount of about 40 mg/day; XL765 is orally administered in amounts of from about 5 mg/day to about 50 mg/day; XL147 is orally administered in amounts of from about 25 mg/day to about 100 mg/day; and combinations thereof.

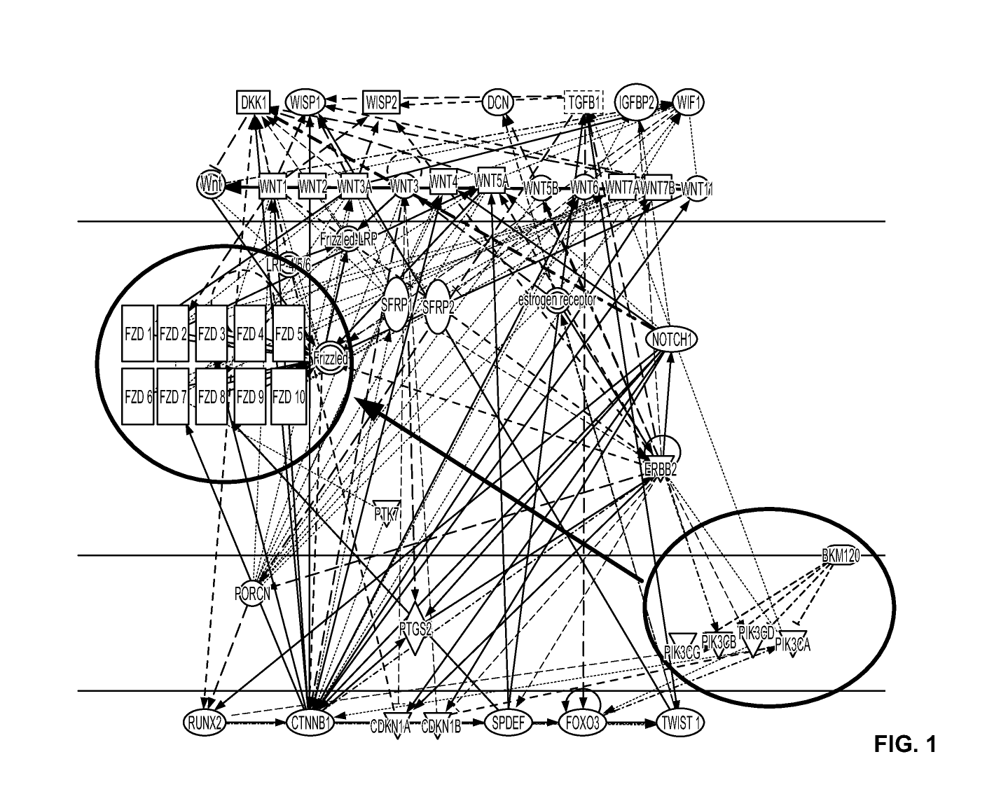

It has been found that inhibition of the PI3K pathway, such as by administration of the PI3K inhibitors described above, leads to compensatory activation of the Wnt (wingless-type MMTV integration site family) signaling pathway (see FIG. 1). Wnt signaling plays a critical role in the embryonic development of a variety of organisms. Particularly, the inhibition of the PI3K pathway leads to upregulation of integral molecules in the Wnt pathway, including the WNT maturation protein, porcupine (PORCN), which is a membrane bound O-acyltransferase required for Wnt palmitoylation, secretion, and biologic activity. As shown in FIG. 1, when Buparlisib is added in silico (circled and in bold), It results in certain components of the PI3K pathway to become inhibited including: phosphatidylinositol 4,5-bisphosphate 3-kinase catalytic subunit alpha isoform (PIK3CA), phosphatidylinositol 4,5-bisphosphate 3-kinase catalytic subunit beta isoform (PIK3CB), phosphatidylinositol 4,5-bisphosphate 3-kinase catalytic subunit delta isoform (PIK3CD), phosphatidylinositol 4,5-bisphosphate 3-kinase catalytic subunit gamma isoform (PIK3CG), protein kinase B (AKT), cyclin-dependent kinase inhibitor 1A gene (CDKNIA), Forkhead box 03 (FOXO3), and others. The in silico addition of Buparlisib also causes certain components of the Wnt signaling pathway to become activated, including: the Frizzled receptors, wingless-type MMTV integration site family, member 5A (WNTSA), wingless-type MMTV integration site family, member 6 (WNT6), wingless-type MMTV integration site family, member 7A (WNT7A), wingless-type MMTV integration site family, member 11 (WNT11), cadherin-associated protein (CTNNB1), low density lipoprotein receptor-related protein (LRP) 1/5/6, and others.

In addition to the PI3K inhibitor, the composition further includes an inhibitor of the Wnt signaling pathway. In particularly suitable embodiments, the composition includes an inhibitor of the WNT protein PORCN. Suitable inhibitors include, for example, 2-[5-methyl-6-(2-methylpyridin-4-yl)pyridin-3-yl]-N-(5-pyrazin-2-ylpyridin-2-yl)acetamide (LGK974 also known as WNT 974); 4-(2-Methyl-4-pyridinyl)-N-[4-(3-pyridinyl)phenyl]benzeneacetamide (Wnt-059); N-(6-Methyl-2-benzothiazolyl)-2-[(3,4,6,7-tetrahydro-4-oxo-3-phenylthieno[3,2-d]pyrimidin-2-yl)thio]-acetamide (IWP-2); N-(5-Phenyl-2-pyridinyl)-2-[(3,4,6,7-tetrahydro-4-oxo-3-phenylthieno[3,2-d]pyrimidin-2-yl)thio]acetamide (IWP-L6); (6S,9aS)-N-benzyl-6-(4-hydroxybenzyl)-8-(naphthalen-1-ylmethyl)-4,7-dioxooctahydro-1H-pyrazino[1,2-a]pyrimidine-1-carboxamide (PRI-724); and combinations thereof. In one embodiment, the composition includes the WNT inhibitor, 2-[5-methyl-6-(2-methylpyridin-4-yl)pyridin-3-yl]-N-(5-pyrazin-2-ylpyridin-2-yl)acetamide (LGK974 also known as WNT 974).

As described herein, proper dosing can be determined by those skilled in the art and can depend on the severity of disease, route of administration, subject body type, subject body weight, age of the subject, and combinations thereof. For orally administered WNT 974, for example, 10 mg q.d. (on prescription) can be administered. As described above, the dosage of orally administered WNT 974 can be increased or decreased from 10 mg q.d. depending on the response of the tumor growth and symptoms associated with adverse and/or side-effects. In one embodiment, a single dosage of orally administered WNT 974 is from about 3 mg/kg to about 30 mg/kg. In other embodiments, Wnt PRI-724 can be administered intravenously in amounts ranging from about 320 m g/m²/day to about 905 mg/m²/day.

In one particularly suitable embodiment, the composition includes a combination of 5-(2,6-di-4-morpholinyl-4-pyrimidinyl)-4-(trifluoromethyl)-2-pyridinamine (buparlisib; BKM-120) and 2-[5-methyl-6-(2-methylpyridin-4-yl)pyridin-3-yl]-N-(5-pyrazin-2-ylpyridin-2-yeacetamide (LGK974; WNT 974). Further, in one embodiment, from about 1 nM to about 100 μM of the composition, including from about 100 nM to about 1 μM of the composition, can be administered to a subject.

In some embodiments, the PI3K and Wnt inhibitors may be provided in a pharmaceutical formulation for medicinal applications, comprising the inhibitors and a pharmaceutically acceptable carrier therefor. Representative pharmaceutical formulations include those suitable for oral, parenteral (including subcutaneous, intradermal, intramuscular and intravenous) and rectal administration. The formulations may be presented in unit dosage form and may be prepared by any of the methods well known in the art of pharmacy. Usually, the formulations are prepared by uniformly and intimately bringing into association the active ingredient(s) with a liquid carrier, or a finely divided solid carrier, or both, and then, if necessary, forming the associated mixture into the desired formulation.

Pharmaceutical formulations suitable for oral administration may be presented as discrete units, such as a capsule, cachet, tablet, or lozenge, each containing a predetermined amount of the active ingredient(s); as a powder or granules; as a solution or a suspension in an aqueous liquid or a non-aqueous liquid such as a syrup, elixir or a draught, or as an oil-in-water liquid emulsion or a water-in-oil liquid emulsion. The formulation may also be a bolus, electuary or paste.

A tablet may be made by compressing or molding a pharmaceutical compound with the pharmaceutically acceptable carrier. Compressed tablets may be prepared by compressing in a suitable machine the active ingredient(s) in a free-flowing form, such as a powder or granules, in admixture with, for example, a binding agent, an inert diluent, a lubricating agent, a disintegrating agent and/or a surface active agent. Molded tablets may be prepared by molding in a suitable machine a mixture of the powdered active ingredient(s) moistened with an inert liquid diluent. The tablets may optionally be coated or scored and may be formulated so as to provide slow or controlled release of the active ingredient(s).

Formulations suitable for parenteral administration include aqueous and non-aqueous sterile injection solutions, and may also include an antioxidant, buffer, a bacteriostat and a solution which renders the composition isotonic with the blood of the recipient, and aqueous and non-aqueous sterile suspensions which may contain, for example, a suspending agent and a thickening agent. The formulations may be presented in single unit-dose or multi-dose containers, and may be stored in a lyophilized condition requiring the addition of a sterile liquid carrier prior to use.

The present disclosure is further directed to methods of administering the compositions for treating cancer, including reducing cancer cell growth, reducing cancer cell migration, reducing metastisis, and/or reducing angiogenesis. Generally, the methods include administering a composition comprising a phosphoinositide 3-kinase (PI3K) inhibitor and a Wnt inhibitor to a subject in need thereof. As used herein, “a subject in need thereof” refers to a subject having or suspected of having a specified disease, disorder, or condition. More particularly, in the present disclosure the methods can be used with a subset of subjects who are suspected of or have been diagnosed with various forms of cancer such as, for example, breast cancer, glioblastoma, prostate cancer, non-small cell lung cancer, colon cancer, melanoma, esophageal cancer, and ovarian cancer. In one particular embodiment, the subject is suspected of having or has been diagnosed with triple negative breast cancer (TNBC).

Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

The compositions of the disclosure are preferably formulated such to include the inhibitors in dosage unit form for ease of administration and uniformity of dosage. The expression “dosage unit form” as used herein refers to a physically discrete unit of agent(s) appropriate for the subject to be treated. It will be understood, however, that the total daily usage of the PI3K and Wnt inhibitors and compositions of the present disclosure (also referred to herein as “therapeutically effective amount”) will be decided by the attending physician within the scope of sound medical judgment. More particularly, as used herein, the phrase “therapeutically effective amount” of the inhibitors of the composition used in the methods of the present disclosure refers to a sufficient amount of the inhibitors to treat cancer, and particularly, triple negative breast cancer, as defined herein, at a reasonable benefit/risk ratio applicable to any medical treatment. It can be understood, however, that the total daily usage of the inhibitors and pharmaceutically acceptable compositions including the inhibitors for use in the methods of the present disclosure can be decided by the attending physician within the scope of sound medical judgment. The specific therapeutically effective dose level for any particular subject can depend upon a variety of factors including reduction in size of the cancer cells being treated and the severity of the cancer; activity of the specific inhibitor(s) employed; the specific pharmaceutically acceptable composition employed; the age, body weight, general health, sex and diet of the subject; the time of administration, route of administration, and rate of excretion of the inhibitor(s) employed; the duration of the treatment; drugs used in combination or coincidental with the specific inhibitor(s) employed; and like factors well-known in the medical arts. For example, it is well within the skill of the art to start doses of the inhibitor(s) at levels lower than required to achieve the desired therapeutic effect and to gradually increase the dosage until the desired effect is achieved.

In certain embodiments, the compositions of the disclosure can be administered orally or parenterally. As described herein, proper dosing can be determined by those skilled in the art and can depend on the severity of disease, route of administration, subject body type, subject body weight, age of the subject, and combinations thereof, to obtain the desired therapeutic effect.

The disclosure will be more fully understood upon consideration of the following non-limiting Examples.

### EXAMPLES

### Example 1

In this Example, the activity of the PI3K pathway was analyzed in TNBC tissues and compared to the pathway activity in normal breast tissues.

Particularly, data from RNA-sequencing of 94 TNBCs compared to 20 normal breast tissues micro-dissected for ductal epithelium was generated as described in Rodovich, et al. Breast Cancer Res Treat 143, 57-68 (2014). Statistically significant differentially expressed genes comparing TNBC versus normal breast tissues (as reported in Radovich et al.) were imported into Ingenuity Pathway Analysis (IPA) for pathway, network, and upstream regulator analyses (Qiagen, Redwood City, Calif., USA).

RNA-sequencing for the Buparlisib treated MDA-MB-231 cell line experiment was performed as follows. To enrich for the non-ribosomal RNA transcriptome, RNA samples were first depleted of ribosomal RNA using the Low Input Ribominus Kit (Life Technologies). ERCC spike-in controls (Pool 1) were introduced for all samples (Life Technologies). RNA libraries were constructed using the Ion Total RNA-Seq Kit for AB Library Builder System (Life Technologies) per manufacturer's instructions. Libraries were barcoded using the IonXpress RNA-Seq Barcode 1-16 Kit (Life Technologies), and libraries were quantified using the Agilent TapeStation 2200 along with the DNA D1K Kit. Libraries were diluted to a concentration of 11 picomolar prior to templating and emulsion PCR using the Ion Template OT2 200 v2 kit along with Ion OneTouch 2 instrument (Life Technologies). Templates were quantified using the IonSphere Quality Control Kit (Life Technologies). Samples were sequenced on an Ion Proton Next-Generation Sequencer using the Ion Proton PI chip and the Ion PI Sequencing 200 v2 kit (Life Technologies). Samples were sequenced using 2 RNA-seq libraries/samples per chip to an average of 30-40 million reads per sample. Reads were mapped to the human genome (hg19) using the STAR algorithm (Dobin et al. Bioinformatics 29, 15-21 (2013)). Aligned BAM files were then imported into Partek Genomics Suite. RPKM values were called in Partek using the NCBI Refseq database as the gene model.

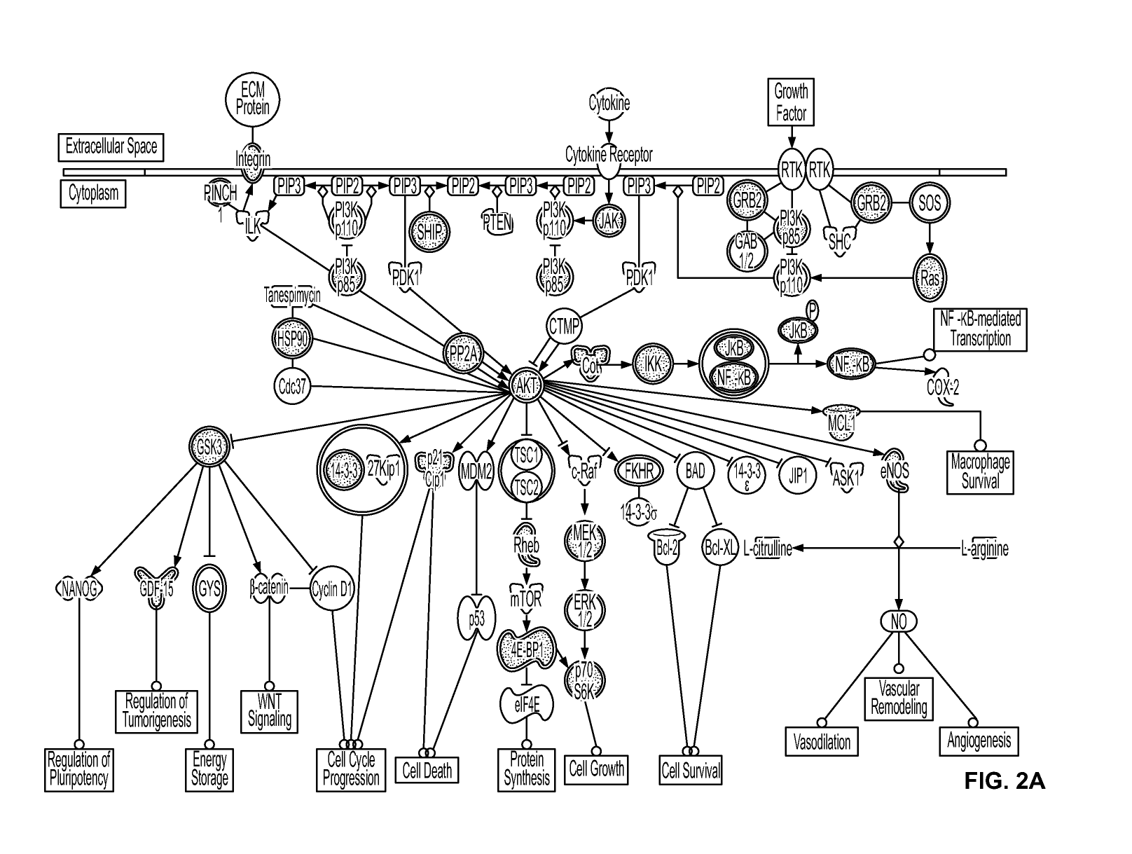

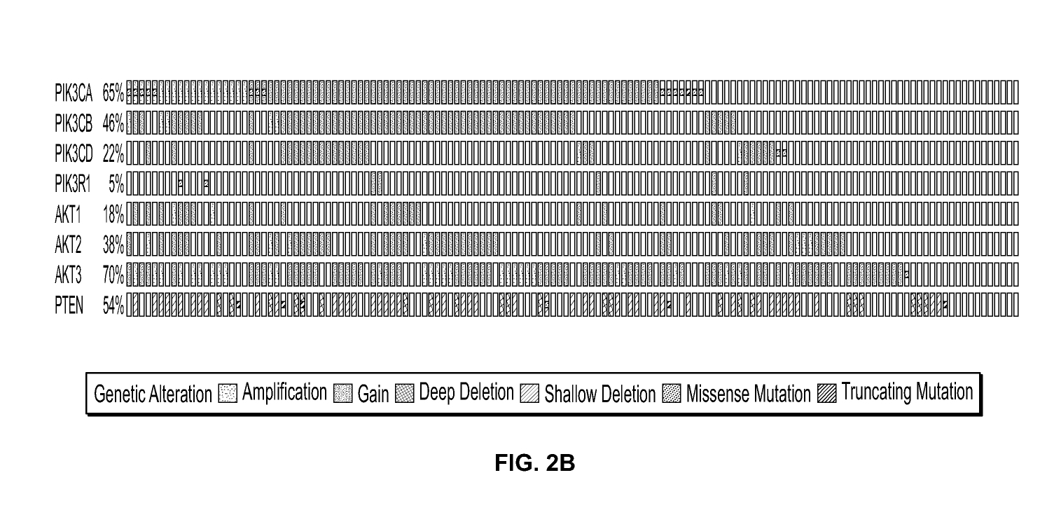

Using the methods above, 3,197 differentially expressed genes were identified in TNBCs as compared to micro-dissected normal breast tissue. To perform drug development studies, network and pathway analysis was then explored to identify key targetable pathways using these differentially expressed genes. From this analysis, over-expression of the PI3K/AKT/mTOR (PI3K) pathway was observed whose components included over-expressed AKT, p21, 4E-BP1, S6 Kinase, and others (FIG. 2A). Congruent with the RNA-seq data, published analyses from The Cancer Genome Atlas (TCGA) also confirmed genomic activation of this pathway even in TNBC samples that do not harbor PIK3CA or PTEN mutations. To further illustrate this, the cBioPortal database (cbioportal.org) was used to mine the TCGA TNBC data, and as shown in FIG. 2B, the majority of TNBCs harbor a genomic aberration in the canonical components of the PI3K, AKT, and/or PTEN genes.

### Example 2

In this Example, the in silico observation of Wnt signaling pathway induction after PI3K inhibition as shown in FIG. 1 was confirmed.

It is well known that single-agent therapy is not always clinically effective, and resistance is common secondary to the activation of compensatory pathways. To this end, the RNA-seq data was analyzed to identify complementary pathways that could be targeted using rational combinations. To do this, an in silico experiment using a new tool called Molecular Activity Predictor (MAP) analysis (a part of the Ingenuity Pathway Analysis 9.0 package) was used. The MAP tool enables the prediction of upstream and/or downstream effects of activation or inhibition of molecules in a network or pathway given a starting set of neighboring molecules with “known” activity or expression. This tool leverages the vast literature library present in Ingenuity Pathway Analysis. As seen in FIG. 1, when Buparlisib is added in silico (circled and in bold), it results in certain components of the PI3K pathway to become inhibited including: PIK3CA, PIK3CB, PIK3CD, PIK3CG and others. The in silico addition of Buparlisib (BKM120) also causes certain components of the Wnt pathway to become activated including: the Wnt ligands (WNTS, WNT6, WNT7, etc), the Frizzled receptors, Beta-Catenin (CTNNB1), LRP1/5/6, and others.

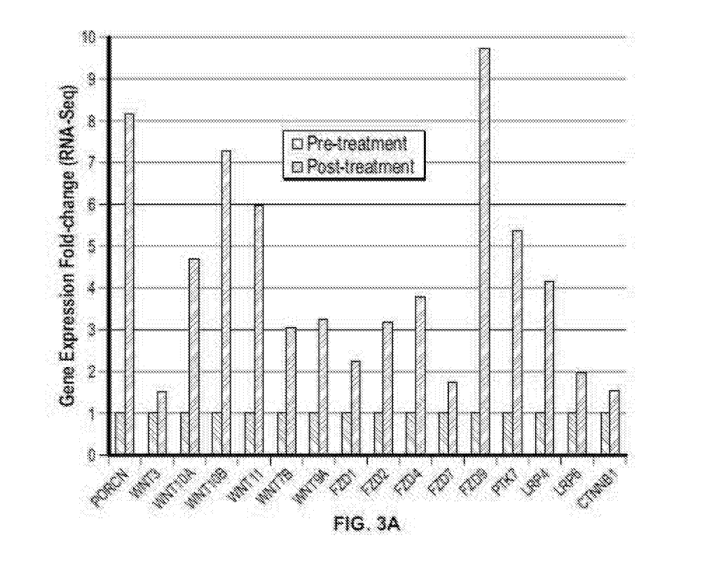

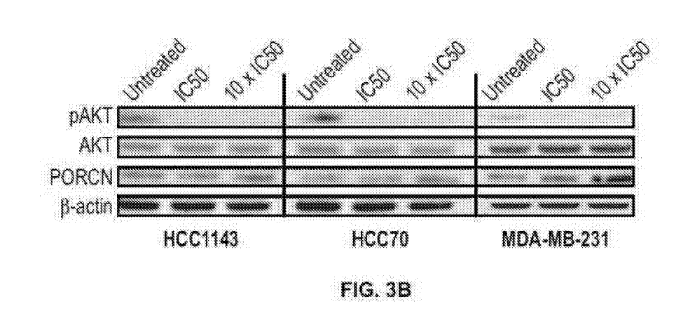

To experimentally confirm the in silico observation of Wnt pathway induction after PI3K inhibition, preliminary data was produced treating the TNBC cell line MDA-MB-231 with Buparlisib, and performing RNA-seq before and after treatment. As shown in FIG. 3A, treatment of MDA-MB-231 cells with Buparlisib results in a significant increase in the expression of Wnt pathway genes. In FIG. 3A, the expression values of a portion of the Wnt pathway genes are shown, including, Porcupine (PORCN), the receptors of Wnt ligands (FZD proteins), Protein Tyrosine Kinase (PTK7), Lipoprotein receptor-related proteins 4 and 6 (LRP4, LRP6), β-catenin (CTNNB1), and several WNT ligands. Of particular interest, the gene Porcupine (PORCN), is approximately 8-fold over-expressed after PI3K inhibition with Buparlisib. Porcupine, is a critical protein involved in Wnt ligand maturation, whose O-acyltransferase activity is required for Wnt ligand pamitoylation and secretion. More importantly, Porcupine is the target of one of only three drugs currently in clinical trial that target the Wnt pathway. WNT974, is an oral small molecule inhibitor of Porcupine, and recently published data has demonstrated potent inhibition of its target and significant inhibition of tumor growth in vivo.

To further confirm the induction of Porcupine after Buparlisib treatment at the protein level, western blotting was used.

For Western Blot analysis, target modulation was assessed using qPCR for Axin2 (a marker of WNT974), and western blot for Porcupine and Phospho-Akt Ser 473 (a marker of Buparlisib activity). qPCR was performed using Taqman assays (Life Technologies). For western blotting, protein was isolated using RIPA buffer and quantified with BCA assay (Thermo Fisher). Protein was run on Bis-Tris gels (Life Technologies) and transferred to PVDF. Primary pAKT-Ser 473 antibody (Cell Signaling Technology, Beverly, Mass.), β-actin (Cell Signaling Technology), and PORCN (Abcam, Cambridge, England) were incubated overnight with subsequent 1 hour incubation with HRP-conjugate secondary antibody. Staining was performed using a chemiluminescent substrate (ThermoFisher). Imaging was performed using an LAS-4000 Luminescent Image Analyzer (Fujifilm, Tokyo, Japan).

Three TNBC cell lines: MDA-MB-231, HCC70, and HCC1143 were treated with either 0, 1 μM or 10 μM Buparlisib. After 72 hours, protein was isolated and PORCN and pAKT expression was observed.

Across the three TNBC cell lines, treatment with Buparlisib resulted in potent inhibition of phospho-AKT as expected (FIG. 3B). Conversely, an increase in Porcupine protein expression after incubation with Buparlisib was observed (FIG. 3B).

### Example 3

In this Example, PI3K & Wnt pathway drug sensitivity and synergy on three TNBC cell lines; Hs578T, HCC70, and MDA-MB-231 were analyzed.

Buparlisib (a pan-PI3K inhibitor), and WNT974 (a Wnt pathway inhibitor, formerly known as LGK974) were obtained under a Material Transfer Agreement with Novartis (Basel, Switzerland). Cells were maintained either in DMEM or RPMI media with 10% FBS, in humidified 5% CO₂ incubators. 10,000 cells per well were seeded in 96-well plates and were dosed with increasing log concentrations (1 nM-100 μM) of Buparlisib and WNT974 as single-agents and in combination. Cells were treated in a 6×6 matrix such that each combination of each dose was tested, with a cascade plot generated from the data. Cell viability was assayed using the Celltiter-Fluor kit (Promega, Madison, Wis.) with fluorescence measured using a Synergy 4 microplate reader (BioTek, Winooski, Vermont). The Chou-Talalay method was used to calculate a Combination Index (CI) score to determine synergism.

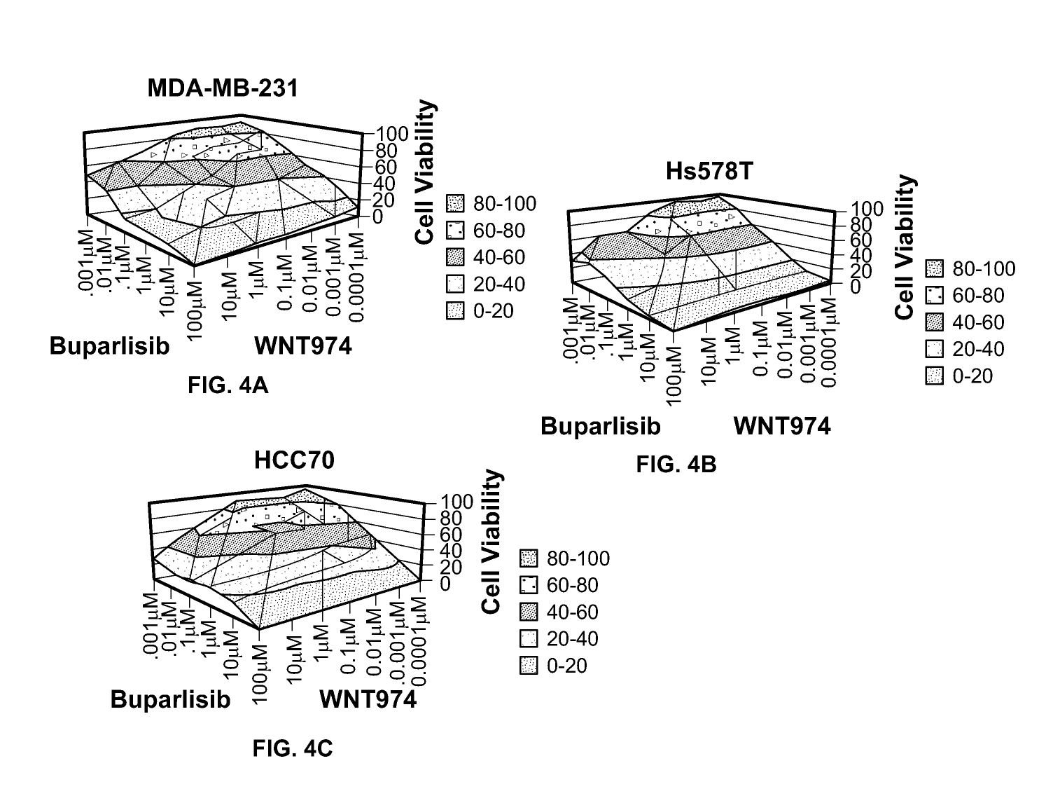

As shown in FIGS. 4A-4C, for MDA-MB-231 and Hs578T cells, a synergistic increase was observed in IC₅₀ at 100 nM concentrations of both Buparlisib and WNT974. An increased IC₅₀ effect was also observed for HCC70 cells at 1 μM concentrations of both Buparlisib and WNT974.

### Example 4

In this Example, an in vivo pharmacokinetic (PK) study was performed.

For pharmacokinetic studies, NOD-scid IL2Rgamma-null (NSG) mice were obtained from the In Vivo Therapeutics (IVT) Core at the Indiana University School of Medicine and given a single dose of Buparlisib, WNT974, or the combination at pre-established concentrations of 30 mg/kg and 3 mg/kg respectively. This experiment employed 45 NSG mice (n=15 for each group). Plasma was drawn from 3 mice from each group at 0.5, 1, 2, 6, and 24 hours post dosage. Buparlisib and WNT974 were quantified in the plasma using high-performance liquid chromatography-mass spectrometry (HPLC-MS/MS). Pharmacokinetic (PK) parameters for Buparlisib and WNT974 including area under the curve (AUC), area under the moment curve (AUMC), and half-life (t½) were estimated using non-compartmental methods with Excel and WinNonlin v 2.0 (Certara, Princeton, N.J.). The maximum plasma concentration (Cₘₐₓ) and time of Cₘₐₓ (tₘₐₓ) was calculated from the data. The systemic clearance (Cl/F, where F=bioavailability) of each drug was calculated from the dose and AUC_{0-∝}The apparent volume of distribution (Vdₛₛ/F) was estimated from the Cl/F and mean residence time. The single-agent pharmacokinetic parameters were compared to the drug combination parameters to determine whether a drug-drug interaction exists.

As shown in Table 1, PK parameters were largely similar when comparing each drug alone or in combination as demonstrated by similar Cmax concentrations, AUCs, clearance rates (Cl/F) and Volumes of Distribution (Vdss/F), suggesting the lack of a drug-drug interaction.

TABLE 1
In vivo single-dose PK measurements of Bubarlisib, WNT974, and the combination.

| Compound | Dosage | Route of | Cₘₐₓ | tₘₐₓ | AUC_{0-∞} | t_{1/2} | Cl/F | Vdₛₛ/F |
|---|---|---|---|---|---|---|---|---|
| Measured | (mg/Kg) | Admin | (ng/mL) | (hours) | (ng*mL⁻¹*hr) | (hours) | (L/hr) | (L) |
| Buparlisib Only | 30 | po | 2303 | 1 | 16536 | 3.0 | 0.034 | 0.15 |
| WNT974 Only | 3 | po | 165 | 1 | 636 | 2.1 | 0.081 | 0.24 |
| Buparlisib + | 30 | po | 2848 | 2 | 15485 | 2.8 | 0.035 | 0.16 |
| (WNT974) | | | | | | | | |
| WNT974 | 3 | po | 143 | 1 | 621 | 3.0 | 0.087 | 0.40 |
| (+Buparlisib) | | | | | | | | |

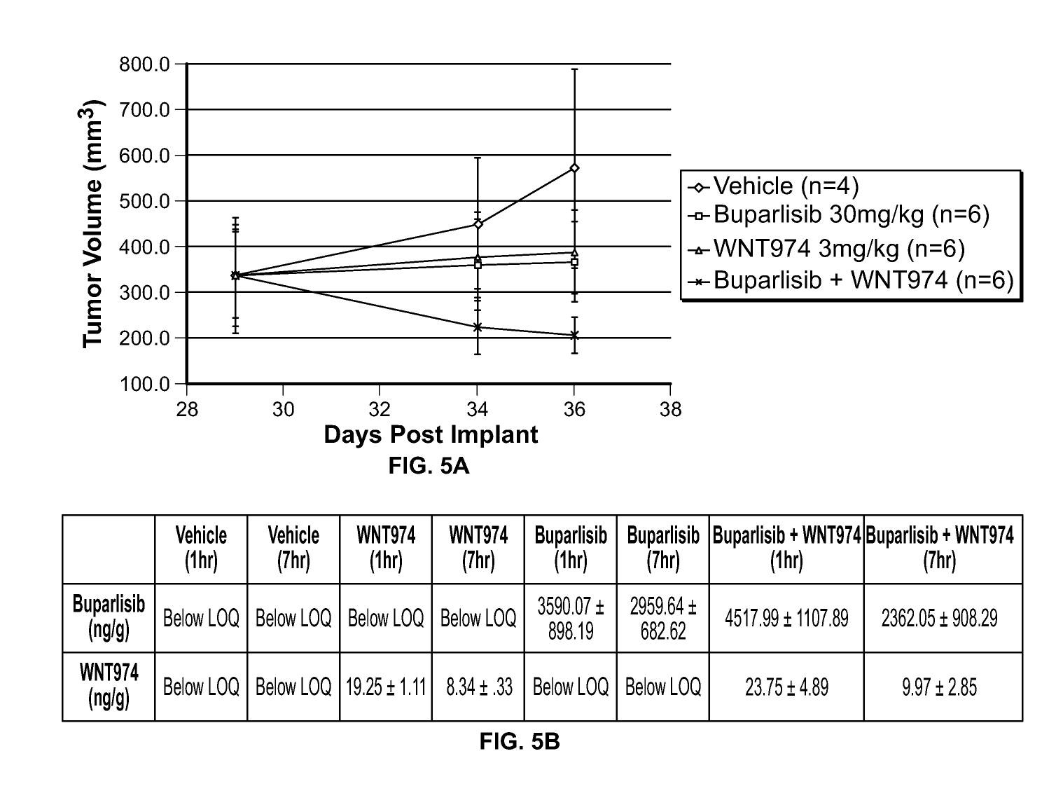

For the pharmacodynamic studies, NSG mice were implanted with TMD-231 TNBC cells into the mammary fat pad of 24 NSG mice (6 mice per group: Vehicle, Buparlisib, WNT974, Buparlisib+WNT974). The TMD-231 cell line is a variant of the MDA-MB-231 cell line that metastasizes to the lung. Tumors were allowed to grow for 29 days to an average size of approximately 350 mm³ prior to dosing. To validate compound-mediated modulation of the PI3K/mTOR and Wnt signaling pathways, mice were dosed once-a-day with vehicle, Buparlisib (30 mg/kg), WNT974 (3 mg/kg), or the combination for 7 days, with intermittent tumor measurements. After only 7 days of treatment, a ˜40% decrease was observed in tumor volume with the combination, while tumors actually grew slightly in the face of single agent therapy, again, giving further observations of synergy (FIG. 5A).

On the seventh day, the mice had plasma drawn, were euthanized, and the tumors were removed. To capture early and late pharmacodynamics effects, half the mice in each group were euthanized with tumors removed at 1 hour after the last dose and the other half at 7 hours. The drugs in the plasma and tumor tissue were quantified as described previously.

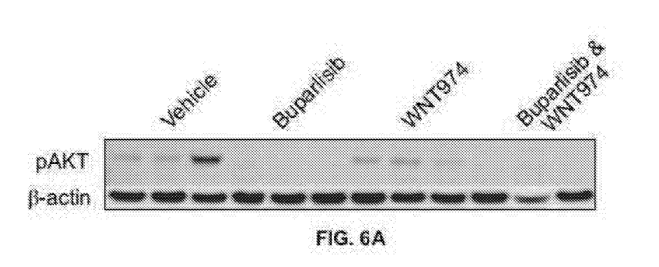

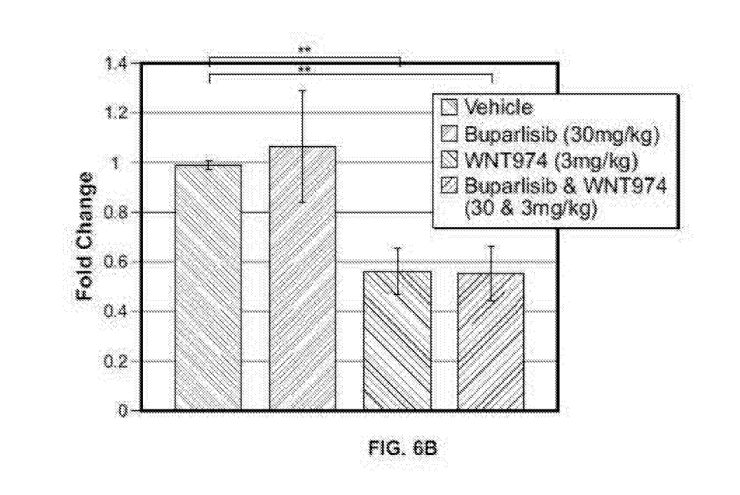

Measurement of Buparlisib and WNT974 concentrations in tumor tissue confirmed that these agents were reaching the tumor (FIG. 5B). To assess molecular target modulation, RNA and protein was isolated from each of the tumors in the PD study and biomarker analysis was performed Inhibition of Phospho-AKT, a marker for the activity of Buparlisib, was observed in the Buparlisib and Buparlisib+WNT974 samples, but not in the vehicle or the WNT974 alone samples. (FIG. 6A) Similarly, decreased AXIN2 RNA levels (a marker of WNT974 activity) was observed in the WNT974 and combination treated tumors, but not the others (FIG. 6B).

To quantify lung metastases, lungs were extracted, fixed in 10% formalin, and processed in paraffin. Five-micrometer sections were H&E stained, deparaffinized, blocked, and stained using primary anti-human Ki67 antibody (DAKO, Carpinteria, Calif.). The Aperio whole slide digital imaging system was used for imaging (Leica, Wetzlar, Germany). Using the Aperio ScanScope CS, 20× images were taken at scan times ranging from 1.5 to 2.25 minutes. The total nuclear labeling index (Ki67) was generated using the Aperio ImageScope standard positive pixel algorithm. The Image Analysis software was used to calculate the percent of positive pixels (brown staining) in one large cross section from each lung.

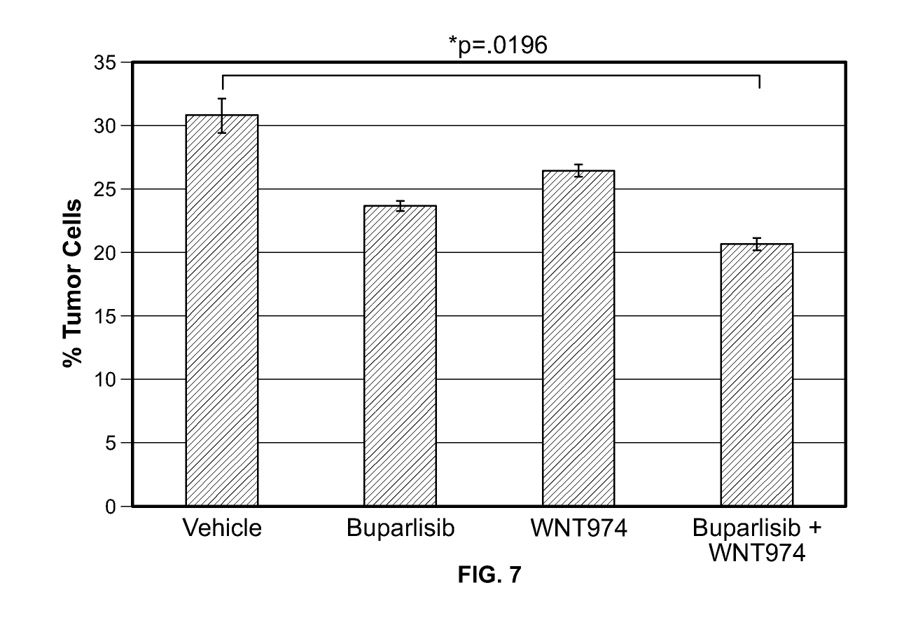

When compared with the control group, Buparlisib and WNT974 as single agents displayed small but non-significant decreases in lung metastases (FIG. 7). The combination treatment, however, resulted in a significant decrease in lung metastatic burden compared to the control group (P=0.0196) (FIG. 7).

### Example 5

In this Example, the efficacy of the combination of Buparlisib and LGK974 (WNT974) against a TNBC patient derived xenograft (PDX) was analyzed.

NSG mice were trochar implanted in the right hind flank with a passage 1-4, 5 mm³ TNBC patient derived xenograft (PDX) (ID: BR0901) at The Jackson Laboratory (The Jackson Laboratory, Bar Harbor, Maine). This tumor was BRCA1 mutated, and had an observed increase in expression in AKT3 and PIK3CA, and a significant decrease in expression of PTEN, demonstrating an active PI3K/AKT pathway. 40 TNBC PDX implanted mice were used (10 mice per group: Vehicle, Buparlisib (30 mg/kg), WNT974 (3 mg/kg), and Buparlisib+WNT974). Tumors were grown to an average of 70-150 mm³ and mice averaged 22.5 grams body weight. Mice were dosed once-a-day for a maximum of 28 days. Tumor measurements were made two times a week. Mice whose tumors reached a size of 1500 mm³ were euthanized per IACUC protocol.

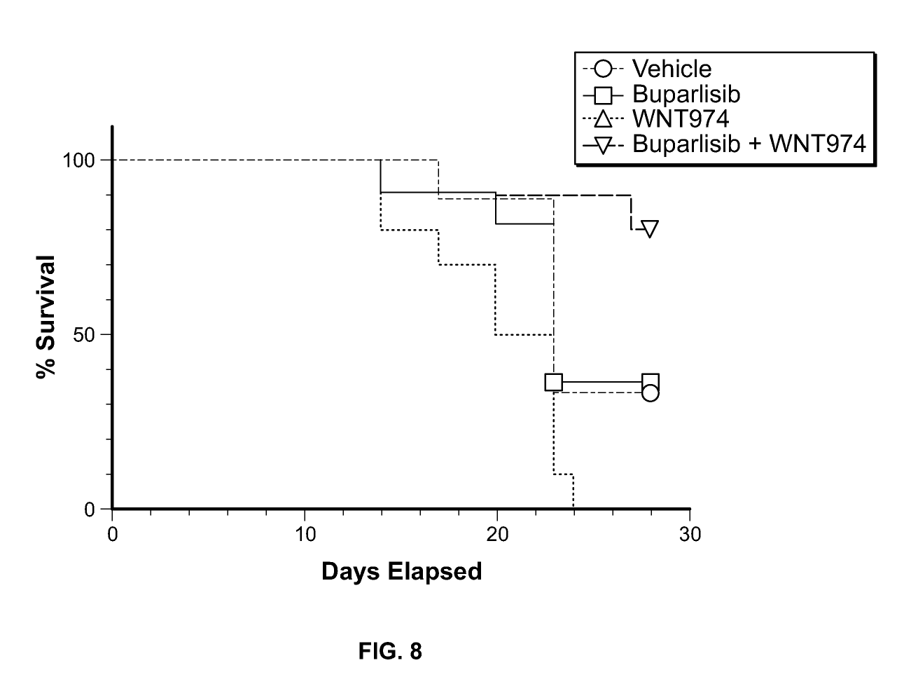

At the end of the experiment, 80% of mice treated with the combination survived to the end of the study (FIG. 8). In comparison, only 40%, 30%, and 0% of mice survived treatment with Buparlisib, Vehicle, and WNT974, respectively (FIG. 8).

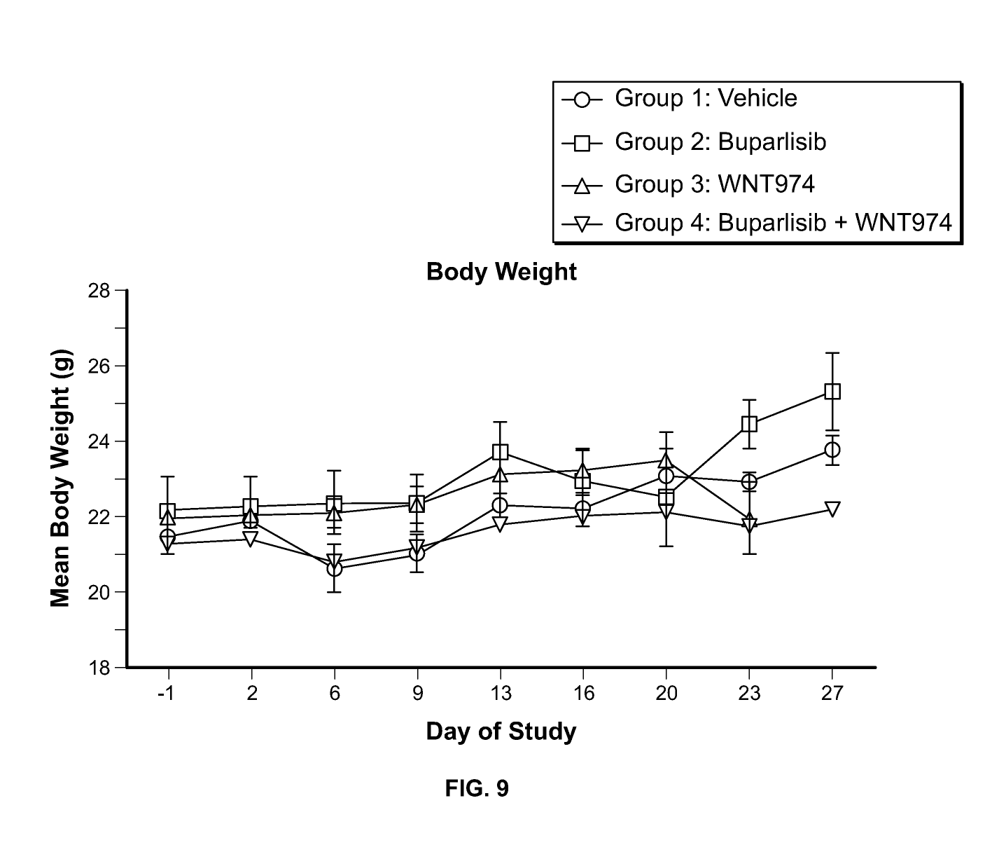

Buparlisib is known to have toxicity in humans (depression, anxiety, pneumonitis, and liver toxicity) that may be exacerbated with the addition of WNT974 (whose most common side effect is dysgeusia). To assess potential toxicity of the combination, the body weights of the mice were measured throughout the experiment. Body weights remained consistent between the four treatment groups with a slight average increase in body weight during the course of experiment (FIG. 9).

Based on the foregoing, a synergistic combination of dual PI3K & Wnt pathway inhibition using Buparlisib+WNT974 against triple-negative breast cancer has been shown.

1. A composition comprising a phosphoinositide 3-kinase (PI3K) inhibitor and a Wnt inhibitor.

2. The composition of claim 1 wherein the PI3K inhibitor is selected from the group consisting of 5-(2,6-di-4-morpholinyl-4-pyrimidinyl)-4-(trifluoromethyl)-2-pyridinamine (buparlisib; B KM-120); 2-(1H-indazol-4-yl)-6-[[4-(methylsulfonyl)-1-piperazinyl]methyl]-4-(4-morpholinyl)-thieno[3 ,2-d]pyrimidine (Pictilisib, GDC-0941); (1E,4S,4aR,5R,6aS ,9aR)-5-(acetyloxy)-1-[(di-2-propen-1-ylamino)methylene]-4,4a, 5,6,6a, 8,9,9a-octahydro-11-hydroxy-4-(methoxymethyl)-4a,6a-dimethyl-cyclopenta[5,6]naphtho[1,2-c]pyran-2,7,10(1H)-trione (PX866); 2-{3-[2-(1-isopropyl-3-methyl-1H-1,2-4-triazol-5-yl)-5,6-dihydrobenzo[f]imidazo[1,2-d][1,4]oxazepin-9-yl]-1H-pyrazol-1-yl}-2-methylpropanamide (taselisib; GDC-0032); 2-methyl-2-[4-(3-methyl-2-oxo-8-quinolin-3-ylimidazo[4,5-c]quinolin-1-yl)phenyl]propanenitrile (dactoblisib; BEZ-235); (2S)-N1-[4-methyl-5-[2-(2,2,2-trifluoro-1,1-dimethylethyl)-4-pyridinyl]-2-thiazolyl]-1,2-pyrrolidinedicarboxamide (alpelisib; BYL-719); (Z)-but-2-enedioic acid; 8-(6-methoxypyridin-3-yl)-3-methyl-1-[4-piperazin-1-yl-3-(trifluoromethyl)phenyl]imidazo[4,5-c]quinolin-2-one (BGT-226); (2S)-1-[4-[[2-(2-aminopyrimidin-5-yl)-7-methyl-4-morpholin-4-ylthieno[3,2-d]pyrimidin-6-yl]methyl]piperazin-1-yl]-2-hydroxypropan-1-one (apitolisib; GDC-0980); N-[4-[[[3-[(3,5-dimethoxyphenyl)amino]-2-quinoxalinyl]amino]sulfonyl]phenyl]-3-methoxy-4-methyl-benzamide (voxtalisib; XL-765); 2-amino-N-[3-[[3-(2-chloro-5-methoxyanilino)quinoxalin-2-yl]sulfamoyl]phenyl]-2-methylpropanamide (pilaralisib; XL-147); 3-(2,4-diaminopteridin-6-yl)phenol (TG100713); 1-[4-(3-ethyl-7-morpholin-4-yltriazolo[4,5-d]pyrimidin-5-yl)phenyl]-3-[4-(4-methylpiperazine-1-carbonyl)phenyl]urea (PKI-402); 5-Fluoro-3-phenyl-2-[(1S)-1-(7H-purin-6-ylamino)propyl]-4(3H)-quinazolinone (idelalisib); and combinations thereof.

3. The composition of claim 1 wherein Wnt inhibitor is an inhibitor of porcupine (PORCN), the inhibitor being selected from the group consisting of 2-[5-methyl-6-(2-methylpyridin-4-yl)pyridin-3-y]-N-(5-pyrazin-2-ylpyridin-2-yl)acetamide (LGK974); 4-(2-Methyl-4-pyridinyl)-N-[4-(3-pyridinyl)phenyl]benzeneacetamide (Wnt-059); N-(6-Methyl-2-benzothiazolyl)-2-[(3,4,6,7-tetrahydro-4-oxo-3-phenylthieno[3,2-d]pyrimidin-2-yl)thio]-acetamide (IWP-2); N-(5-Phenyl-2-pyridinyl)-2-[(3,4,6,7-tetrahydro-4-oxo-3-phenylthieno[3 ,2-d]pyrimidin-2-yl)thio]acetamide (IWP-L6); (6S ,9aS)-N-benzyl-6-(4-hydroxybenzyl)-8-(naphthalen-1-ylmethyl)-4,7-dioxooctahydro-1H-pyrazino[1,2-a]pyrimidine-1-carboxamide (PRI-724); and combinations thereof.

4. The composition of claim 1 comprising 5-(2,6-di-4-morpholinyl-4-pyrimidinyl)-4-(trifluoromethyl)-2-pyridinamine (buparlisib; B KM-120) and 2-[5-methyl-6-(2-methylpyridin-4-yl)pyridin-3-yl]-N-(5-pyrazin-2-ylpyridin-2-yl)acetamide (LGK974).

5. The composition of claim 1 further comprising a pharmaceutically acceptable carrier.

6. A method for treating cancer in a subject in need thereof, the method comprising administering a composition comprising a phosphoinositide 3-kinase (PI3K) inhibitor and a Wnt inhibitor to the subject.

7. The method of claim 6 wherein the PI3K inhibitor is selected from the group consisting of 5-(2,6-di-4-morpholinyl-4-pyrimidinyl)-4-(trifluoromethyl)-2-pyridinamine (buparlisib; BKM-120); 2-(1H-indazol-4-yl)-6-[[4-(methylsulfonyl)-1-piperazinyl]methyl]-4-(4-morpholinye-thieno[3,2-d]pyrimidine (Pictilisib, GDC-0941); (1E,4S,4aR,5R,6aS,9aR)-5-(acetyloxy)-1-[(di-2-propen-1-ylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866); 2-{3-[2-(1-isopropyl-3-methyl-1H-1,2-4-triazol-5-yl)-5,6-dihydrobenzo[f]imidazo[1,2-d][1,4]oxazepin-9-yl]-1H-pyrazol-1-yl}-2-methylpropanamide (taselisib; GDC-0032); 2-methyl-2-[4-(3-methyl-2-oxo-8-quinolin-3-ylimidazo[4,5-c]quinolin-1-yl)phenyl]propanenitrile (dactoblisib; BEZ-235); (2S)-N1-[4-methyl-5-[2-(2,2,2-trifluoro-1,1-dimethylethyl)-4-pyridinyl]-2-thiazolyl]-1,2-pyrrolidinedicarboxamide (alpelisib; BYL-719); (Z)-but-2-enedioic acid;8-(6-methoxypyridin-3-yl)-3-methyl-1-[4-piperazin-1-yl-3-(trifluoromethyl)phenyl]imidazo[4,5-c]quinolin-2-one (BGT-226); (2S)-1-[4-[[2-(2-aminopyrimidin-5-yl)-7-methyl-4-morpholin-4-ylthieno[3,2-d]pyrimidin-6-yl]methyl]piperazin-1-yl]-2-hydroxypropan-1-one (apitolisib; GDC-0980); N-[4-[[[3-[(3,5-dimethoxyphenyl)amino]-2-quinoxalinyl]amino]sulfonyl]phenyl]-3-methoxy-4-methyl-benzamide (voxtalisib; XL-765); 2-amino-N-[3-[[3-(2-chloro-5-methoxyanilino)quinoxalin-2-yl]sulfamoyl]phenyl]-2-methylpropanamide (pilaralisib; XL-147); 3-(2,4-diaminopteridin-6-yl)phenol (TG100713); 1-[4-(3-ethyl-7-morpholin-4-yltriazolo[4,5-d]pyrimidin-5-yl)phenyl]-3-[4-(4-methylpiperazine-1-carbonyl)phenyl]urea (PKI-402); 5-Fluoro-3-phenyl-2-[(1S)-1-(7H-purin-6-ylamino)propyl]-4(3H)-quinazolinone (idelalisib); and combinations thereof.

8. The method of claim 6 wherein the Wnt inhibitor is an inhibitor of porcupine (PORCN), the inhibitor being selected from the group consisting of 2-[5-methyl-6-(2-methylpyridin-4-yl)pyridin-3-yl]-N-(5-pyrazin-2-ylpyridin-2-yl)acetamide (LGK974); 4-(2-Methyl-4-pyridinyl)-N-[4-(3-pyridinyl)phenyl]benzeneacetamide (Wnt-059); N-(6-Methyl-2-benzothiazolyl)-2-[(3,4,6,7-tetrahydro-4-oxo-3-phenylthieno[3,2-d]pyrimidin-2-yl)thio]-acetamide (IWP-2); N-(5-Phenyl-2-pyridinyl)-2-[(3,4,6,7-tetrahydro-4-oxo-3-phenylthieno[3,2-d]pyrimidin-2-yl)thio]acetamide (IWP-L6); (6S,9aS)-N-benzyl-6-(4-hydroxybenzyl)-8-(naphthalen-1-ylmethyl)-4,7-dioxooctahydro-1H-pyrazino[1,2-a]pyrimidine-1-carboxamide (PRI-724); and combinations thereof.

9. The method of claim 6 wherein the composition comprises 5-(2,6-di-4-morpholinyl-4-pyrimidinyl)-4-(trifluoromethyl)-2-pyridinamine (buparlisib; B KM-120) and 2-[5-methyl-6-(2-methylpyridin-4-yl)pyridin-3-yl]-N-(5-pyrazin-2-ylpyridin-2-yl)acetamide (LGK974).

10. The method of claim 6 wherein the composition further comprises a pharmaceutically acceptable carrier.

11. The method of claim 6 wherein the composition is administered by a method selected from the group consisting of oral administration, parenteral administration, rectal administration, and combinations thereof.

12. A method for treating triple-negative breast cancer in a subject in need thereof, the method comprising administering a composition comprising a phosphoinositide 3-kinase (PI3K) inhibitor and a Wnt inhibitor to the subject.

13. The method of claim 12 wherein the PI3K inhibitor is selected from the group consisting of 5-(2,6-di-4-morpholinyl-4-pyrimidinyl)-4-(trifluoromethyl)-2-pyridinamine (buparlisib; BKM-120); 2-(1H-indazol-4-yl)-6-[[4-(methylsulfonyl)-1-piperazinyl]methyl]-4-(4-morpholinyl)-thieno[3,2-d]pyrimidine (Pictilisib, GDC-0941); (1E,4S,4aR,5R,6aS,9aR)-5-(acetyloxy)-1-[(di-2-propen-1-ylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866); 2-{3-[2-(1-isopropyl-3-methyl-1H-1,2-4-triazol-5-yl)-5,6-dihydrobenzo[f]imidazo[1,2-d][1,4]oxazepin-9-yl]-1H-pyrazol-1-yl}-2-methylpropanamide (taselisib; GDC-0032); 2-methyl-2-[4-(3-methyl-2-oxo-8-quinolin-3-ylimidazo[4,5-c]quinolin-1-yl)phenyl]propanenitrile (dactoblisib; BEZ-235); (2S)-N1-[4-methyl-5-[2-(2,2,2-trifluoro-1,1-dimethylethyl)-4-pyridinyl]-2-thiazolyl]-1,2-pyrrolidinedicarboxamide (alpelisib; BYL-719); (Z)-but-2-enedioic acid;8-(6-methoxypyridin-3-yl)-3-methyl-1-[4-piperazin-1-yl-3-(trifluoromethyl)phenyl]imidazo [4,5-c]quinolin-2-one (BGT-226); (2S)-1-[4-[[2-(2-aminopyrimidin-5-yl)-7-methyl-4-morpholin-4-ylthieno[3,2-d]pyrimidin-6-yl]methyl]piperazin-1-yl]-2-hydroxypropan-1-one (apitolisib; GDC-0980); N-[4-[[[3-[(3,5-dimethoxyphenyl)amino]-2-quinoxalinyl]amino]sulfonyl]phenyl]-3-methoxy-4-methyl-benzamide (voxtalisib; XL-765); 2-amino-N-[3-[[3-(2-chloro-5-methoxyanilino)quinoxalin-2-yl]sulfamoyl]phenyl]-2-methylpropanamide (pilaralisib; XL-147); 3-(2,4-diaminopteridin-6-yl)phenol (TG100713); 1-[4-(3-ethyl-7-morpholin-4-yltriazolo[4,5-d]pyrimidin-5-yl)phenyl]-3-[4-(4-methylpiperazine-1-carbonyl)phenyl]urea (PKI-402); 5-Fluoro-3-phenyl-2-[(1S)-1-(7H-purin-6-ylamino)propyl]-4(3H)-quinazolinone (idelalisib); and combinations thereof.

14. The method of claim 12 wherein the Wnt inhibitor is an inhibitor of porcupine (PORCN), the inhibitor being selected from the group consisting of 2-[5-methyl-6-(2-methylpyridin-4-yl)pyridin-3-yl]-N-(5-pyrazin-2-ylpyridin-2-yl)acetamide (LGK974); 4-(2-Methyl-4-pyridinyl)-N-[4-(3-pyridinyl)phenyl]benzeneacetamide (Wnt-059); N-(6-Methyl-2-benzothiazolyl)-2-[(3,4,6,7-tetrahydro-4-oxo-3-phenylthieno[3,2-d]pyrimidin-2-yl)thio]-acetamide (IWP-2); N-(5-Phenyl-2-pyridinyl)-2-[(3,4,6,7-tetrahydro-4-oxo-3-phenylthieno[3,2-d]pyrimidin-2-yl)thio]acetamide (IWP-L6); (6S,9aS)-N-benzyl-6-(4-hydroxybenzyl)-8-(naphthalen-1-ylmethyl)-4,7-dioxooctahydro-1H-pyrazino[1,2-a]pyrimidine-1-carboxamide (PRI-724); and combinations thereof.

15. The method of claim 12 wherein the composition comprises 5-(2,6-di-4-morpholinyl-4-pyrimidinyl)-4-(trifluoromethyl)-2-pyridinamine (buparlisib; BKM-120) and 2-[5-methyl-6-(2-methylpyridin-4-yl)pyridin-3-yl]-N-(5-pyrazin-2-ylpyridin-2-yl)acetamide (LGK974).

16. (canceled)

17. The method of claim 12 wherein the composition is administered by a method selected from the group consisting of oral administration, parenteral administration, rectal administration, and combinations thereof.

18-23. (canceled)

24. The method of claim 6 wherein the method of treating cancer comprises reducing cancer cell growth in the subject.

25. The method of claim 24 wherein the composition comprises 5-(2,6-di-4-morpholinyl-4-pyrimidinyl)-4-(trifluoromethyl)-2-pyridinamine (buparlisib; BKM-120) and 2-[5-methyl-6-(2-methylpyridin-4-yl)pyridin-3-yl]-N-(5-pyrazin-2-ylpyridin-2-yl)acetamide (LGK974).

26. The method of claim 24 wherein the composition further comprises a pharmaceutically acceptable carrier.

27. The method of claim 24 wherein the composition is administered by a method selected from the group consisting of oral administration, parenteral administration, rectal administration, and combinations thereof.
